(19) United States
(12) Patent Application Publication
(10) Pub. No.: US 2024/0008461 A1
(43) Pub. Date: Jan. 11, 2024

(54) CRUCIAN CARP (CARASSIUS AURATUS) STRAIN WITHOUT INTERMUSCULAR BONES AND BREEDING METHOD THEREOF

(71) Applicant: HEILONGJIANG RIVER FISHERIES RESEARCH INSTITUTE OF CHINESE ACADEMY OF FISHERY SCIENCES, Harbin, Heilongjiang (CN)

(72) Inventors: Youyi KUANG, Harbin, Heilongjiang (CN); Xianhu ZHENG, Harbin, Heilongjiang (CN); Guangxiang TONG, Harbin, Heilongjiang (CN); Zhipeng SUN, Harbin, Heilongjiang (CN); Dingchen CAO, Harbin, Heilongjiang (CN); Ting YAN, Harbin, Heilongjiang (CN); Huan XU, Harbin, Heilongjiang (CN); Le DONG, Harbin, Heilongjiang (CN); Xiaoxing YANG, Harbin, Heilongjiang (CN); Tianqi LIU, Harbin, Heilongjiang (CN); Tan ZHANG, Harbin, Heilongjiang (CN); Tingting ZHANG, Harbin, Heilongjiang (CN); Xiaowen SUN, Harbin, Heilongjiang (CN)

(21) Appl. No.: 18/035,797

(22) PCT Filed: Jan. 7, 2022
(86) PCT No.: PCT/CN2022/070608

§ 371 Date: May 8, 2023

(30) Foreign Application Priority Data
Apr. 30, 2021 (CN) 202110478410.7

(51) Int. Cl. A01K 67/027; C12N 9/22

(52) U.S. Cl. CPC A01K 67/0276; C12N 9/22; C12N 2310/20; A01K 2217/075; A01K 2227/40; A01K 2267/02

(57) ABSTRACT

The disclosure provides a method for breeding Carassius auratus strains without intermuscular bones, which comprises designing knockout target sites for the two copies of the bmp6, bmp6a and bmp6b, in the Carassius auratus genome, and then obtaining F2 generation individuals from a homozygous line with bmp6a and bmp6b double gene mutation through two rounds of gene knockout and screening, and then propagating by using the F2 generation individuals from the homozygous line with bmp6a and bmp6b double gene mutation to form a new Carassius auratus strain with intermuscular bone-deficient. Strains of Carassius auratus with less than 20 intermuscular bones and without intermuscular bones are obtained by the method of the present disclosure.

### CROSS REFERENCE TO RELATED APPLICATION

This application is a national stage application of International Patent Application No. PCT/CN2022/070608, filed on Jan. 7, 2022, which claims the priority of Chinese Patent Application No. 202110478410.7, entitled “Crucian carp (Carassius auratus) strain without intermuscular bones and breeding method thereof” filed in China National Intellectual Property Administration on Apr. 30, 2021, both of which are incorporated by reference herein in its entirety as part of the present application.

### REFERENCE TO SEQUENCE LISTING

A computer readable ASCII file entitled “HLPCTP20230201217-sequence_listing.txt”, that was created on May 4, 2023, with a file size of about 6,743 bytes, contains the sequence listing for this application, has been filed with this application, and is hereby incorporated by reference in its entirety.

### Technical Field

The present disclosure relates to a breeding method of Carassius auratus strain without intermuscular bones, belonging to the technical field of aquaculture breeding methods.

### Background Art

Cyprinid is one of the main aquaculture fish in China, with an annual output of about 20 million tons in China, providing nearly ⅓ of the high-quality animal protein for the Chinese people. However, because of lots of intermuscular bones in cyprinid fishes, it is inconvenient for people to eat, and may even lead to physical injuries such as getting stuck in the throat. At the same time, the processing of fish products (such as fish balls, etc.) is also hindered. Therefore, the genetic improvement of intermuscular bones in cyprinid fishes has become one of the most important targets of aquaculture genetic breeding.

At present, there are some reports on selection and breeding of intermuscular bones. For example, Xiao-feng Xu et al.^{[1]} found a normally grown and developed intermuscular bone-deficient mutant in the gynogenetic group of grass carp, but no mutant population of grass carp with intermuscular bone-deficient or less intermuscular bone was obtained as reported. Brazilian scientists^{[2]} screened a population lacking intermuscular bones in Colossoma macropomum using X-rays, but no population with less intermuscular bone or intermuscular bone-deficient in the subsequent breeding program was obtained^{[3]}. Guo et al.^{[4]} conducted a hybrid experiment between Megalobrama terminalis♀ and Culter alburnus♂, and showed that the number of intermuscular bones (127) in the hybrid progeny of Megalobrama terminalis♀ and Culter alburnus♂ was smaller than that of the male parent (137), greater than that of the female parent (124), but significantly fewer than the number of intermuscular bones in the hybrid progeny (133) of Megalobrama amblycephala♀ and Culter alburnus♂, which indicated that varieties with less intermuscular bones could be obtained by hybrid breeding methods, and an invention patent (publication number of CN107347747A) was applied then. Li Ling et al.^{[5]} reported that the number of intermuscular bones in the artificial cultivation of hybrid Carassius auratus was less than that of the wild Carassius auratus, and the number of intermuscular bones in the Carassius auratus could be reduced by artificial cultivation and hybrid breeding. Bao-long Bao et al. invented a method for thicking the intermuscular bones by knocking out the MSTN gene^{[9-12]}. By knocking out the scxa gene in zebrafish (Danio rerio), Gao Zexia et al. obtained mutants with more than 70% reduction of the number of intermuscular bones, but also caused dysplasia of other bones such as ribs^{[6-8]}, and the applications in other Cyprinoid fish has not been reported. In conclusion, although there are some methods to reduce the number of intermuscular bones in cyprinid fish, there is still no new variety/strain without intermuscular bones.

Crucian carp (Carassius auratus) is one of the main aquaculture varieties in China, with an annual production of about 3 million tons. Its meat is delicious and popular among people. At the same time, the quality of Carassius auratus has been affected due to the intermuscular bones (the average number of intermuscular bones in different varieties of Carassius auratus is about 71 to 84), thus, intermuscular bones are also an important target for their genetic breeding. Although it has been reported that the number of intermuscular bones in Carassius auratus can be reduced by hybrid breeding and artificial selection, no strain or variety with more than 50% reduction in the number of intermuscular bones has been found.

### REFERENCES

[1] Xu, X., Zheng, J., Qian, Y. & Luo, C. Normally grown and developed intermuscular bone-deficient mutant in grass carp, Ctenopharyngodon idellus. Chin Sci Bulletin Chin Version 60, 52 6 (2015).
[2] Perazza, C. A. et al. Lack of intermuscular bones in specimens of Colossoma macropomum: An unusual phenotype to be incorporated into genetic improvement programs. Aquaculture 472, 57 60 (2017).
[3] Stokstad, E. Tomorrow's catch. Science 370, 902-905 (2020).
[4] Guo, H.-H. et al. Comparative analysis of the growth performance and intermuscular bone traits in F1 hybrids of black bream (Megalobrama terminalis) (♀)×topmouth culter (Culter alburnus) (♂). Aquaculture (2018) doi:10.1016/j.aquaculture. 2018.03.037.
[5] Li, L. et al. Comparative analysis of intermuscular bones in fish of different ploidies. Sci China Life Sci 56, 341-350 (2013).
[6] Nie, C. et al. Loss of scleraxis leads to distinct reduction of mineralized intermuscular bone in zebrafish. Aquac Fish 6, 169-177 (2021).
[7] Kague, E. et al. Scleraxis genes are required for normal musculoskeletal development and for rib growth and mineralization in zebrafish. Faseb J 33, 9116-9130 (2019).
[8] CN110684777A (Huazhong Agricultural University, Publication date 2020 Jan. 14);
[9] CN111560401A (Shanghai Ocean University, Publication date 2020 Aug. 21);
[10] CN111549030A (Shanghai Ocean University, Publication date 2020 Aug. 18);
[11] CN111549031A (Shanghai Ocean University, Publication date 2020 Aug. 18);
[12] CN111500581A (Shanghai Ocean University, Publication date 2020 Aug. 7);
[13] Chen, Z. et al. De novo assembly of the goldfish (Carassius auratus) genome and the evolution of genes after whole-genome duplication. Sci Adv 5, eaav0547 (2019).
[14] Tong, G. et al. De novo assembly and characterization of the Hucho taimen transcriptome. Ecol Evol 8, 1271 1285 (2018).
[15] Fiume, M. et al. CRISPResso2 provides accurate and rapid genome editing sequence analysis. Nat Biotechnol 37, 224-226 (2019).

### SUMMARY

In order to perform genetic improvement of intermuscular bones in Carassius auratus, the present disclosure proposes a method for breeding Carassius auratus with fewer intermuscular bones or without intermuscular bones by knocking out the bmp6 gene.

The method for breeding Carassius auratus strains without intermuscular bones of the present disclosure carries out the breeding of the Carassius auratus strain without intermuscular bones according to the following steps:

Designing knockout target sites for two copies of bmp6 gene in the Carassius auratus genome, bmp6a and bmp6b, respectively, and then obtaining F2 generation individuals from a homozygous line with bmp6a and bmp6b double gene mutation through two rounds of gene knockout and screening, and then using the F2 generation individuals from the homozygous line with bmp6a and bmp6b double gene mutation to propagate to form a new Carassius auratus strain without intermuscular bone;

Among them, mixing the sgRNA corresponding to (bmp6a), (bmp6b) or (bmp6a and bmp6b) with Cas9 protein and microinjecting a mixture obtained into Carassius auratus embryos in the single cell stage, to perform the first round of gene knockout to construct the F0 generation population, culturing the F0 generation population for 3 to 5 months followed by PIT labeling and DNA extraction, then sequencing to determine the alleles and mutation rates of somatic mutations of Carassius auratus, selecting the F0 generation individuals with the somatic mutation rate of more than 95% as parents to prepare 0-generation fertilized eggs;

Mixing the sgRNAs corresponding to (bmp6a), (bmp6b) or (bmp6a and bmp6b) with Cas9 protein and microinjecting a mixture obtained into 0-generation fertilized eggs to perform the second round of gene knockout to construct the F1 generation population, culturing the F1 generation population for 3 to 5 months followed by PIT labeling and DNA extraction, then sequencing to determine the alleles and mutation rates of somatic mutations of Carassius auratus, selecting the F1 generation individuals from a somatic bmp6a and bmp6b double gene mutant line with the somatic mutation rate of more than 95% as parents for reproduction to construct F2 generation, then selecting F2 generation individuals from a homozygous line with bmp6a and bmp6b double gene mutation.

Carassius auratus varieties with less than 20 intermuscular bones and without intermuscular bones are obtained by the method of the present disclosure.

Since the Carassius auratus genome has undergone a fourth round of genome duplication event^{[}13], there are two copies of the bmp6 gene in the Carassius auratus genome.

### BRIEF DESCRIPTION OF THE DRAWINGS

FIG. 1 is a comparison picture of bone staining observation of a typical individual in Example 1, and arrows in the figure refer to intermuscular bones;

FIG. 2 is an X-ray diagram of wild-type Carassius auratus bone in Example 1, and the arrow in the figure indicates intermuscular bones;

FIG. 3 is an X-ray diagram of caudal bones of a mutant in Example 1, from which it can be seen that no intermuscular bone is present in the caudal muscle tissue;

FIG. 4 is an X-ray diagram of trunk bones of a mutant in Example 1, from which it can be seen that no intermuscular bone is present in back muscle tissue of trunk;

FIGS. 5A, 5B and 5C are diagrams showing the sequencing results of exon 1 of the new Carassius auratus strain bmp6a in Example 1; CAA-1, CAA-2, and CAA-3 are target sites of exon 1 of bmp6a; WT represents wild type, the number in sequence number is the PIT labeling number of mutant, and the letter followed by PIT labeling number is the label of mutant allele;

FIGS. 6A, 6B and 6C are diagrams showing the sequencing results of exon 1 of the new Carassius auratus strain bmp6b in Example 1; CAA-7, CAA-8, and CAA-9 are target sites of exon 1 of bmp6b; WT represents wild type, the number in sequence number is the PIT labeling number of mutants, and the letter followed by PIT labeling number is the label of mutant allele;

FIG. 7 is a comparison diagram of exon 1 protein sequences of the new Carassius auratus strain bmp6a in Example 1; WT represents wild type, the number in sequence number is the PIT labeling number of mutants, and the letter followed by PIT labeling number is the label of mutant allele;

FIG. 8 is a comparison diagram of exon 1 protein sequences of the new Carassius auratus strain bmp6b in Example 1; WT represents wild type, the number in sequence number is the PIT labeling number of mutants, and the letter followed by PIT labeling number is the label of mutant allele;

FIG. 9 is a micro-CT image of bones of wild-type and the new Carassius auratus strain in Example 1;

FIG. 10 is a viviperception diagram by X-ray of wild type, typical individuals with few intermuscular bones and typical individuals without intermuscular bones in Example 1, arrows indicate intermuscular bones.

### DETAILED DESCRIPTION OF THE EMBODIMENTS

The present disclosure is further described in detail below in conjunction with examples. The following examples are intended to illustrate the present disclosure, but not to limit the scope herein.

The experimental methods used in the following examples are conventional methods unless otherwise specified. The materials, reagents, methods and instruments used, unless otherwise specified, are conventional materials, reagents, methods and instruments in the art, which are commercially available to those skilled in the art.

Embodiment 1: The breeding method of Carassius auratus strain without intermuscular bones of the present embodiment:

The target sites are designed to knock out two copies of bmp6 gene in the Carassius auratus genome, bmp6a and bmp6b, respectively, and then F2 generation individuals from a homozygous line with bmp6a and bmp6b double gene mutation are obtained through two rounds of gene knockout and screening, and then the F2 generation individuals from the homozygous line with bmp6a and bmp6b double gene mutation are propagated to form a new Carassius auratus strain without intermuscular bone;

Among them, the sgRNAs corresponding to (bmp6a), (bmp6b) or (bmp6a and bmp6b) are mixed with Cas9 protein, and the obtained mixture is microinjected into Carassius auratus embryos in the single cell stage, the first round of gene knockout is performed to construct the F0 generation population, and then the F0 generation population is cultured for 3 to 5 months followed by PIT labeling and DNA extraction then the alleles and mutation rates of somatic mutations of Carassius auratus are determined by sequencing, and the F0 generation individuals with the somatic mutation rate of more than 95% are selected as parents to prepare 0-generation fertilized eggs;

The sgRNAs corresponding to (bmp6a), (bmp6b) or (bmp6a and bmp6b) are mixed with Cas9 protein, and the obtained mixture is microinjected into 0-generation fertilized eggs, the second round of gene knockout is performed to construct the F1 generation population, and then the F1 generation population is cultured for 3 to 5 months followed by PIT labeling and DNA extraction, then the alleles and mutation rates of the somatic mutations of Carassius auratus are determined by sequencing, and the F1 generation individuals from a somatic bmp6a and bmp6b double gene mutant line with the somatic mutation rate of more than 95% are selected as parents for reproduction to construct an F2 generation, then F2 generation individuals from a homozygous line with bmp6a and bmp6b double gene mutation are selected.

Among them, the DNA extraction and sequencing method is as follows: 100 μL of lysis solution is added (the lysis solution comprises 0.5 mg/mL proteinase K, 10 mM Tris (pH 8.0), 50 mM KCl, 0.3% Tween 20, 0.3% NP40) to 0.3-0.5cm² fin clip samples; the lysis methods is: the PCR instrument is set at 55° C. for 6 h, 98° C. for 10 min for lysis; after lysis, the obtained product is mixed well, then centrifuged at 1000-2000 rpm for 2 min, and the supernatant is taken as a PCR amplification template.

PCR amplification: 2 pairs of primers are used for PCR amplification, of which, 1 pair of primers is the target site primers (the primer sequences used to amplify the target fragment are shown in Table 2 and concatenated by the M13 universal primer sequence at 5′ end), and the other pair of primers is index primers (the M13 universal primer sequence and the index base sequence at the 5′ end used to identify the sample). A two-step PCR amplification is performed. The PCR amplification volume in the first step is 10 L, including 1 μL of genomic DNA supernatant, 0.5 μL of 1 μM forward and reverse target primers, and 5 μL of 2×Dream Taq Master Mix (Thermo Fisher, CA, USA), the volume is supplemented to 10 μL with enzyme-free water; PCR program: 95° C. for 3 min; 10 cycles of 95° C. for 30 s, 60° C. for 30 s and 72° C. for 30 s; 72° C. for 2 min; PCR amplification in step 2 is that 0.5 μL of 5 μM forward and reverse index primers are added to the PCR amplification product obtained in step 1, the PCR program is set to 95° C. for 2 min; 6 cycles of 95° C. for 30 s, 58° C. for 30 s and 72° C. for 30 s; 15 cycles of 95° C. for 30 s, 72° C. for 30 s; 72° C. for 2 min.

After PCR amplification, the PCR products are mixed in equal amounts, and a DNA sequencing library is constructed with the TrueSeq Sample Preparation Kit, and is sequenced using Illumina MiSeq sequencing platform with 300 bp Pair-End mode. The data obtained by high-throughput sequencing are first used to identify amplification loci and samples using the method described by Tong et al.^{[14]}. The sequenced data of each sample in each PCR product is analyzed using the CRISPResso2^{[15]} program to determine the alleles and mutation rates of somatic mutations.

Among them, Sanger sequencing is used for mutant detection: PCR amplification is performed on the samples using the primers shown in Table 2, and the volume is set to 25 μL: 2 μL of genomic DNA supernatant, 1 μL of 10 μM forward and reverse target primers, 12.5 μL of 2×Dream Taq Master Mix (Thermo Fisher, CA, USA), the volume is supplemented to 25 μL with enzyme-free water; PCR program is 95° C. for 3 min; 35 cycles of 95° C. for 30 s, 60° C. for 30 s, 72° C. for 30 s; 72° C. for 5 min. PCR products are detected by 1.5% agarose gel electrophoresis. After detection, the products are purified and recovered, TA clone is performed, 2 μL of PCR products are taken after colony PCR amplification, and detected with 8% polypropylene, and the colonies with different band sizes from the control are selected for Sanger sequencing.

Embodiment 2: The difference between this embodiment and Embodiment 1 is that the knockout target sites of the bmp6 gene are shown in Table 1 in this embodiment.

Knockout target sites of bmp6 gene

| | sgRNAs | Genome | | | |
|---|---|---|---|---|---|
| Gene | Number | coordinates | Exon | Target sites sequence | sgRNA forward primer |
| bmp6a | CAA1 | NC_039266.1: | Exon1 | GGTAGCAGTTCTGCACTAGACGG | TTCTAATACGACTCACTATAGGTAGCAG |
| | | 5784476 | | (SEQ ID NO: 1) | TTCTGCACTAGAGTTTTAGAGCTAGA |
| | | | | | (SEQ ID NO: 12) |
| | CAA2 | NC_039266.1: | Exon1 | CCAGCGGAGGTTGCGGACTCAGG | TTCTAATACGACTCACTATAGCCAGCGG |
| | | 5784523 | | (SEQ ID NO: 2) | AGGTTGCGGACTCGTTTTAGAGCTAGA |
| | | | | | (SEQ ID NO: 13) |
| | CAA3 | NC_039266.1: | Exon1 | GAAAGAGATTCTGTCCATACTGG | TTCTAATACGACTCACTATAGGAAAGAG |
| | | 5784562 | | (SEQ ID NO: 3) | ATTCTGTCCATACGTTTTAGAGCTAGA |
| | | | | | (SEQ ID NO: 14) |
| | CAA4 | NC_039266.1: | Exon2 | TCTTTATGGTGTCTTCTCTGTGG | TTCTAATACGACTCACTATAGTCTTTAT |
| | | 5815487 | | (SEQ ID NO: 4) | GGTGTCTTCTCTGGTTTTAGAGCTAGA |
| | | | | | (SEQ ID NO: 15) |
| | CAA5 | NC_039266.1: | Exon2 | GGCCTCTCCCTCTGGAATCTGGG | TTCTAATACGACTCACTATAGGCCTCTC |
| | | 5815527 | | (SEQ ID NO: 5) | CCTCTGGAATCTGTTTTAGAGCTAGA |
| | | | | | (SEQ ID NO: 16) |
| | CAA6 | NC_039266.1: | Exon2 | TGCAGAATTCAGGATCTACAAGG | TTCTAATACGACTCACTATAGTGCAGAA |
| | | 5815558 | | (SEQ ID NO: 6) | TTCAGGATCTACAGTTTTAGAGCTAGA |
| | | | | | (SEQ ID NO: 17) |
| bmp6b | CAA7 | NC_039291.1: | Exon1 | GGCAGGTAGTAGTTCTGTACTGG | TTCTAATACGACTCACTATAGGCAGGTA |
| | | 8127537 | | (SEQ ID NO: 7) | GTAGTTCTGTACGTTTTAGAGCTAGA |
| | | | | | (SEQ ID NO: 18) |
| | CAA8 | NC_039291.1: | Exon1 | AGCCCAACTTCATTCATCGGAGG | TTCTAATACGACTCACTATAGAGCCCAA |
| | | 8127477 | | (SEQ ID NO: 8) | CTTCATTCATCGGGTTTTAGAGCTAGA |
| | | | | | (SEQ ID NO: 19) |
| | CAA9 | NC_03929.1: | Exon1 | GAAAGAGATCCTGTCCATACTGG | TTCTAATACGACTCACTATAGGAAAGAG |
| | | 8127425 | | (SEQ ID NO: 9) | ATCCTGTCCATACGTTTTAGAGCTAGA |
| | | | | | (SEQ ID NO: 20) |
| | CAA10 | NC_03929.1: | Exon2 | GGCCTCTCCCTCTGGAATCTGGG | TTCTAATACGACTCACTATAGGCCTCTC |
| | | 8097264 | | (SEQ ID NO: 10) | CCTCTGGAATCTGTTTTAGAGCTAGA |
| | | | | | (SEQ ID NO: 21) |
| | CAA11 | NC_039291.1: | Exon2 | AGCAGAATTCAGGATCTACAAGG | TTCTAATACGACTCACTATAGAGCAGAA |
| | | | | (SEQ ID NO: 11) | TTCAGGATCTACAGTTTTAGAGCTAGA |
| | | | | | (SEQ ID NO: 22) |

Others are the same as Embodiment 1.

Primers for PCR amplification

| | | | | Size of |
|---|---|---|---|---|
| | Amplified | | | amplified |
| Primer name | region | Forward primer sequence | Reverse primer sequence | fragment |
| CAA-imb1a-E1 | bmp6a | ATCACAGACTGGATGTAAAGATGA | CATGAAGAGGGGTGCTGAA | 250 bp |
| | exon1 | (SEQ ID NO: 23) | (SEQ ID NO: 24) | |
| CAA-imb1a-E2 | bmp6a | GTTCGTCAAGCACACTAAAACT | CTTTCTGGATGTTCCCCAA | 250 bp |
| | exon2 | (SEQ ID NO: 25) | (SEQ ID NO: 26) | |
| CAA-imb1b-E1 | bmp6b | TCCAGCATGAAGAGGGGTG | CTGGATGTAAAGATGACAAGCA | 247 bp |
| | exon1 | (SEQ ID NO: 27) | (SEQ ID NO: 28) | |
| CAA-imb1b-E2 | bmp6b | ACAGGCAGCAGTTGACTTG | GCTTTCACCACAGAGAAGACA | 322 bp |
| | exon2 | (SEQ ID NO: 29) | (SEQ ID NO: 30) | |

Embodiment 3: The difference between this embodiment and Embodiment 1 or 2 is that: the sgRNA forward primer shown in Table 1 and the sgRNA reverse primer with the sequence of 5′-GATCCGCACCGACTCGGTGCCACTTTTTCAAGTTGATAACGGACTAGCCTT ATTTTAACTTGCTATTTCTAGCTCTAAAAC-3′ (SEQ ID NO:31) are used to synthesize sgRNA in vitro. The sgRNA synthesis volume in vitro is: 2.5 μL of 10 μM sgRNA forward and reverse primers, 25 μL of 2×Dream Taq Master Mix (ThermoFisher, CA, USA), and sgRNA synthesis volume is supplemented to 50 μL with enzyme-free water; a total of 100 μL is amplified in 2 tubes. The sgRNA synthesis procedure in vitro is: denaturation at 95° C. for 3 min, followed by 30 cycles of 95° C. for 30 s, 58° C. for 30 s, 72° C. for 30 s; extension at 72° C. for 5 min; then the obtained amplified product is purified and recovered, followed by an in vitro transcription, and 30 μL of reaction volume is established for each target site: 1 μg of sgRNA PCR recovered product, 10 μL of NTP Buffer Mix, 2 μL of T7 RNA Polymerase Mix, the reaction volume is supplemented to 30 μL with enzyme-free water; transcription was performed at 37° C. for 4 h, 20 μL of enzyme-free water is added after the reaction, and 2μL of DNase I was added after mixing. Digestion at 37° C. for 15 min to remove DNA. Others are the same as the embodiments 1 or 2.

The in vitro PCR products of sgRNA are detected by 1.5% agarose gel electrophoresis, and the length of products is 120 bp. After detection, the PCR products are purified and recovered with a PCR product purification and recovery kit (Exygen), and the concentration is determined with a Qubit 3 kit (Thermo Fisher, CA, USA) for use. The recovery concentration of sgRNA PCR products is in a range of 100 to 160 ng/μL.

The in vitro transcribed sgRNAs are purified and recovered with an RNA purification kit (Qiagen). The concentration of the recovered product is determined by using a Qubit 3 kit (Thermo Fisher, CA, USA) and the recovered product is stored in a −80 ° C. refrigerator until use. The recovery concentration of in vitro transcribed sgRNA is in a range of 800 to 3000 ng/μL.

Embodiment 4: The difference between this embodiment and Embodiment 1 or 2 or 3 is that the microinjection method in this embodiment is: the sgRNA synthesized in vitro and Cas9 protein (NEB M0646, MA, USA) are mixed in a molar ratio of 3:1, a mixture obtained is incubated at room temperature for 10 min, then 25% phenol red is added, and the obtained product is injected into Carassius auratus embryos in the single cell stage; among them, the target site sgRNAs on each exon are mixed in equal amounts and injected with a final concentration no less than 50 ng/μL of each sgRNA, the control group is injected with 25% phenol red, and the injection volume of each fertilized egg is 1 nL±0.02 nL. Others are the same as the Embodiment 1, 2 or 3.

### Example 1

Breeding method of Carassius auratus strain without intermuscular bone: The target sites were designed to knock out the two copies of bmp6 gene in the Carassius auratus genome, bmp6a and bmp6b, respectively, and then F2 generation individuals from a homozygous line with bmp6a and bmp6b double gene mutation were obtained through two rounds of gene knockout and screening, and then the F2 generation individuals from the homozygous line with bmp6a and bmp6b double gene mutation were propagated to form a new Carassius auratus strain without intermuscular bones;

Among them, the sgRNAs corresponding to (bmp6a), (bmp6b) or (bmp6a and bmp6b) were mixed with Cas9 protein, and the obtained mixture was microinjected into Carassius auratus embryos in the single cell stage, the first round of gene knockout was performed to construct the F0 generation population, and then the F0 generation population was cultured for 3 to 5 months followed by PIT labeling and DNA extraction, then the alleles and mutation rates of somatic mutations of Carassius auratus was determined by sequencing, and the F0 generation individuals with the somatic mutation rate of more than 95% were selected as parents to prepare 0-generation fertilized eggs;

The sgRNAs corresponding to (bmp6a), (bmp6b) or (bmp6a and bmp6b) were mixed with Cas9 protein, and the obtained mixture was microinjected into the 0-generation fertilized eggs, the second round of gene knockout was performed to construct the F1 generation population, and then the F1 generation population was cultured for 3 to 5 months followed by PIT labeling and DNA extraction, then the alleles and mutation rates of the somatic mutations of Carassius auratus were determined by sequencing, and the F1 generation individuals from a somatic bmp6a and bmp6b double gene mutant line with the somatic mutation rate of more than 95% were selected as parents for reproduction to construct a F2 generation, then F2 generation individuals from a homozygous line with bmp6a and bmp6b double gene mutation were selected.

Among them, the DNA extraction and sequencing method was as follows: 100 μL of lysis solution was added (the composition of the lysate is 0.5 mg/mL proteinase K, 10 mM Tris (pH 8.0), 50 mM KCl, 0.3% Tween 20, 0.3% NP40) to 0.3-0.5 cm² fin clip samples; the lysis methods was: the PCR instrument was set at 55° C. for 6 h, 98° C. for 10 min for lysis; after lysis, the obtained product was mixed well, then centrifuged at 1000-2000 rpm for 2 min, and the supernatant was taken as the PCR amplification template. PCR amplification: 2 pairs of primers were used for PCR amplification, of which, 1 pair of primers was the target site primers (the primer sequences used to amplify the target fragment as shown in Table 2 and the 5′-end M13 universal primer sequence), and the other pair of primers were index primers (the M13 universal primer sequence and the index base sequence at the 5′ end used to identify the sample). PCR amplification was performed in 2 steps. The PCR amplification volume in the first step was 10 μL, including 1 μL of genomic DNA supernatant, 0.5 μL of 1 μM forward and reverse target primers, and 50 μL of 2×Dream Taq Master Mix (Thermo Fisher, CA, USA), the volume was supplemented to 10 μL with enzyme-free water; PCR program: 95° C. for 3 min; 10 cycles of 95° C. for 30 s, 60° C. for 30 s and 72° C. for 30 s; 72° C. for 2 min; PCR amplification in step 2 is that 0.5 μL of 5 μM forward and reverse index primers were added to the PCR amplification product obtained in step 1, the PCR program was set to 95° C. for 2 min; 6 cycles of 95° C. for 30 s, 58° C. for 30 s and 72° C. for 30 s; 15 cycles of 95° C. for 30 s, 72° C. for 30 s; 72° C. for 2 min. After PCR amplification, the PCR products were mixed in equal amounts, and a DNA sequencing library was constructed with the TrueSeq Sample Preparation Kit, and was sequenced by using Illumina MiSeq sequencing platform with 300 bp Pair-End mode. The data obtained by high-throughput sequencing were first used to identify amplification loci and samples using the method described by Tong et al (Tong et al. 2018). The sequenced data of each sample in each PCR product was analyzed using the CRISPResso2 (Fiume et al. 2019) program to determine the alleles and mutation rates of somatic mutations.

Among them, Sanger sequencing was used for mutant detection: PCR amplification was performed on the samples using the primers shown in Table 2, and the system was set to 25 μL: 2 μL of genomic DNA supernatant, 1 μL of 10 μM forward and reverse target primers, 12.5 μL of 2×Dream Taq Master Mix (Thermo Fisher, CA, USA), the volume was supplemented to 25 μL with enzyme-free water; PCR program was 95° C. for 3 min; 35 cycles of 95° C. for 30 s, 60° C. for 30 s, 72° C. for 30 s; 72° C. for 5 min. PCR products were detected by 1.5% agarose gel electrophoresis. After detection, the products were purified and recovered, TA clone was performed, 2 μL of PCR products were taken after colony PCR amplification, and detected with 8% polypropylene, and the colonies with different size bands from the control were selected for Sanger sequencing.

Among them, the knockout target sites of the bmp6 gene were shown in Table 1.

The sgRNA forward primer was shown in Table 1 and the sgRNA reverse primer with the sequence of 5′-GATCCGCACCGACTCGGTGCCACTTTTTCAAGTTGATAACGGACTAGCCTT ATTTTAACTTGCTATTTCTAGCTCTAAAAC-3′ (SEQ ID NO:31) were used to synthesize sgRNA in vitro. The sgRNA synthesis volume in vitro was: 2.5 μL of 10 μM sgRNA forward and reverse primers, 25 μL of 2×Dream Taq Master mix (Thermo Fisher, CA, USA), the volume was supplemented to 50 μL with enzyme-free water; a total of 100 μL was amplified in 2 tubes. The sgRNA synthesis procedure in vitro was: denaturation at 95° C. for 3 min, followed by 30 cycles of 95° C. for 30 s, 58° C. for 30 s, 72° C. for 30 s; extension at 72° C. for 5 min; then the obtained amplified product was purified and recovered, followed by an in vitro transcription, and 30 μL of reaction volume was established for each target site: 1 μg of sgRNA PCR recovered product, 10 μL of NTP Buffer Mix, and 2 μL of T7 RNA Polymerase Mix, the volume was supplemented to 30 μL with enzyme-free water; transcription was performed at 37° C. for 4 h, 20 μL of enzyme-free water was added after the reaction, and 2 μL of DNase I was added after mixing. Digestion at 37° C. for 15 min to remove DNA.

In vitro sgRNA PCR products were detected by 1.5% agarose gel electrophoresis, and the length of products was 120 bp. After detection, the sgRNA PCR products were purified and recovered with a PCR product purification and recovery kit (Exygen), and the concentration was determined with a Qubit 3 kit (Thermo Fisher, CA, USA) for use. The recovery concentration of the sgRNA PCR products was in a range of 100 to 160 ng/μL.

The in vitro transcribed sgRNAs were purified and recovered with an RNA purification kit (Qiagen). The concentration of the recovered product was determined by using a Qubit 3 kit (Thermo Fisher, CA, USA) and the recovered product was stored in a −80 ° C. refrigerator until use. The recovery concentration of in vitro transcribed sgRNA was in a range of 800 to 3000 ng/μL.

The microinjection method was as follows: sgRNA synthesized in vitro and Cas9 protein (NEB M0646, MA, USA) were mixed at a molar concentration ratio of 3:1, a mixture obtained was incubated at room temperature for 10 min, then 25% phenol red was added, and the obtained product was injected into Carassius auratus embryos in the single cell stage; among them, the target site sgRNAs on each exon were mixed in equal amounts and injected with a final concentration no less than 50 ng/μL of each sgRNA, the control group was injected with 25% phenol red, and the injection volume of each fertilized egg is 1 nL±0.02 nL.

The diploid Carassius auratus used in this example came from the Hulan Experiment Station of the Heilongjiang River Fisheries Research Institute, Chinese Academy of Fishery Sciences. The parents fish were cultured in a 700 m² fish pond, and were injected with oxytocin drugs (4 μg/kg luteinizing releasing hormone analogue, LRH-A2; 1 mg/k DOM, 100 units/kg chorionic gonadotropin HCG) for spawn. After artificial fertilization, the obtained fertilized eggs were incubated in a hatching tank of 24 cm×20 cm×24 cm, and the hatching water temperature was 22° C.-23° C. After hatching, larvae were fed with Artemia salina 4 times/d after the yolk was absorbed. The larvae were moved to an outdoor 500 m² pond to culture when they were cultured in a hatching tank to the size of 2 cm-3 cm. Artificial diet was fed in the fish fingerling breeding stage, 2 times/d.

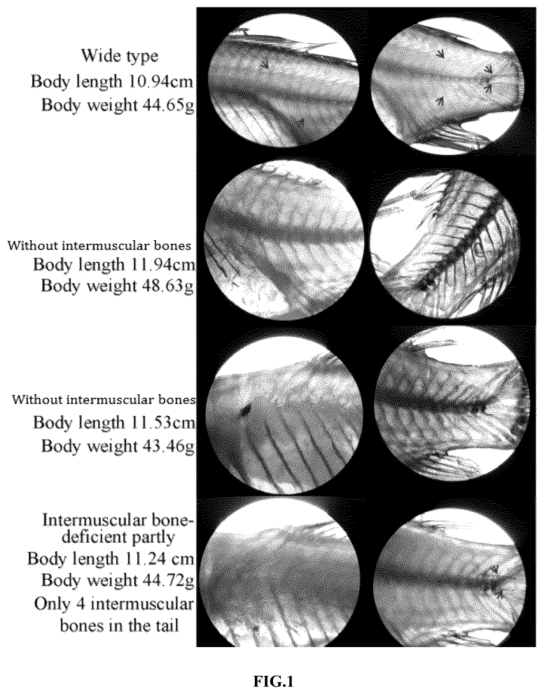

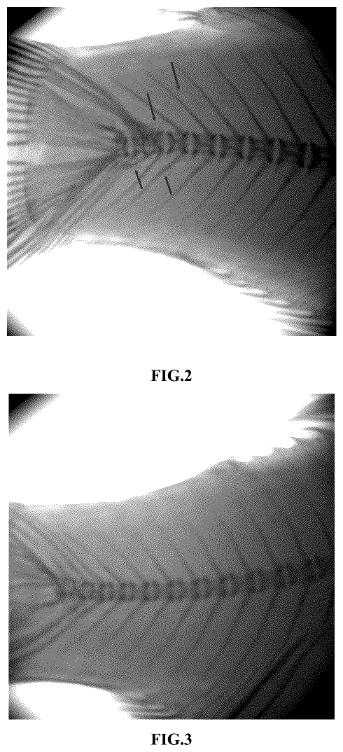

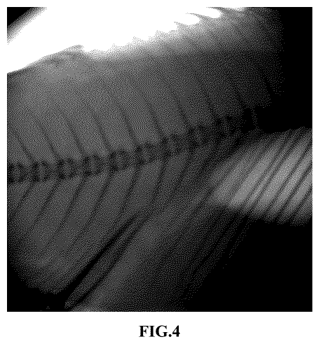

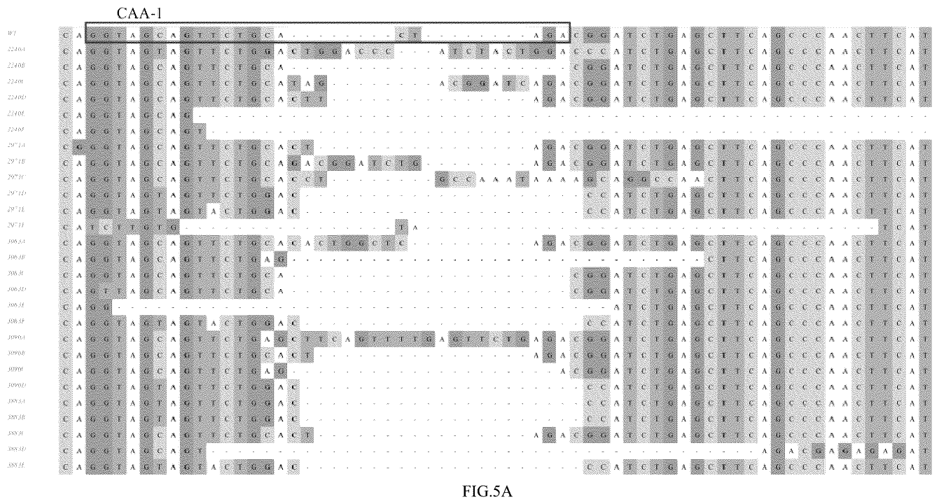

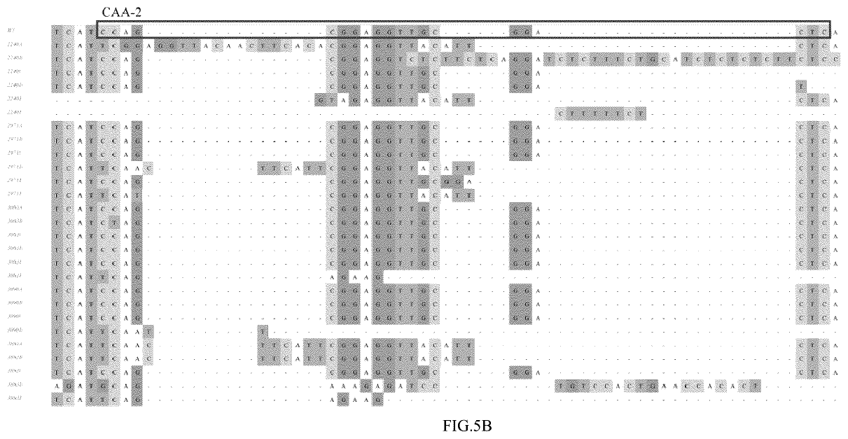

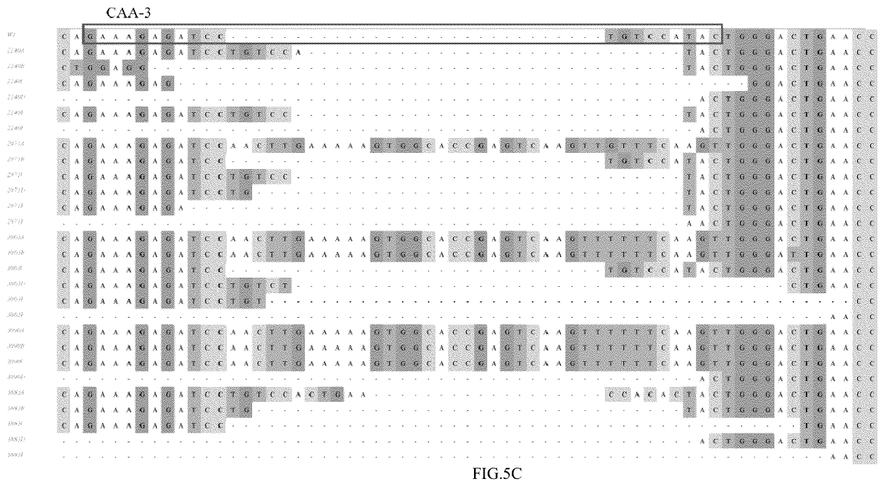

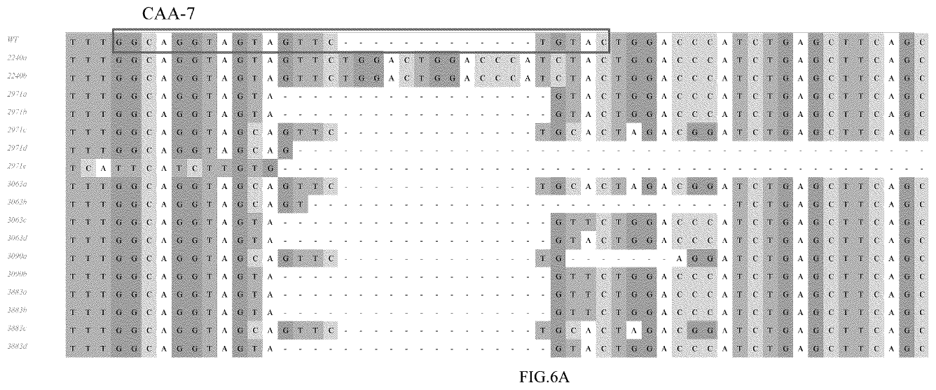

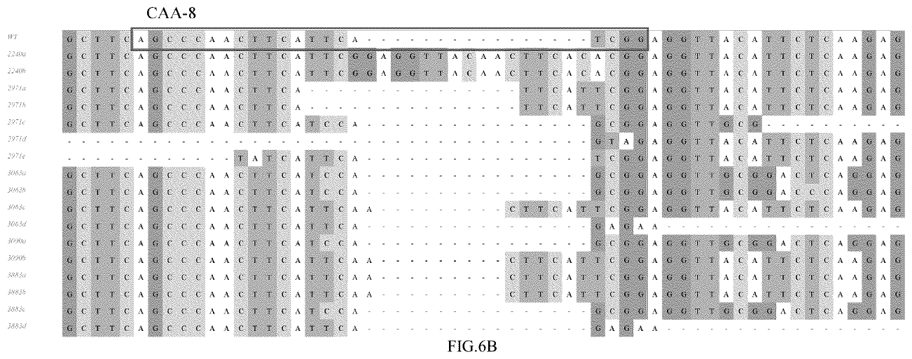

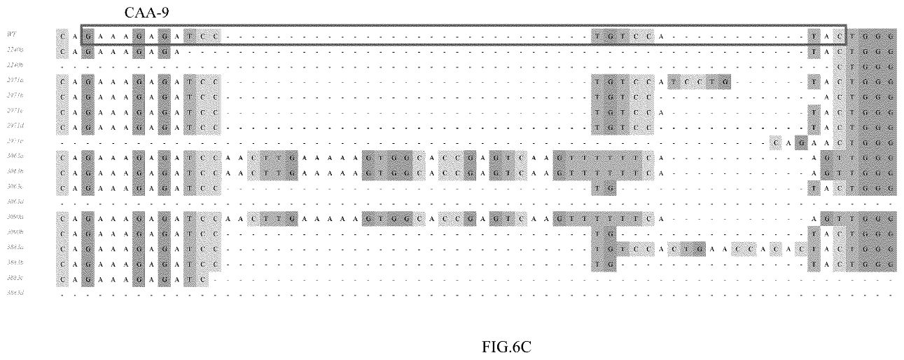

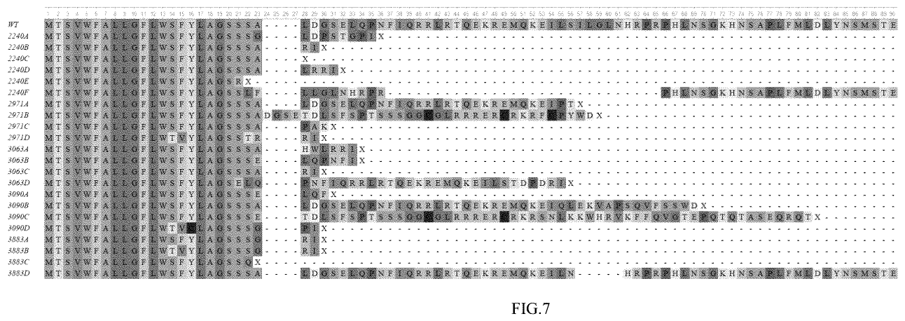

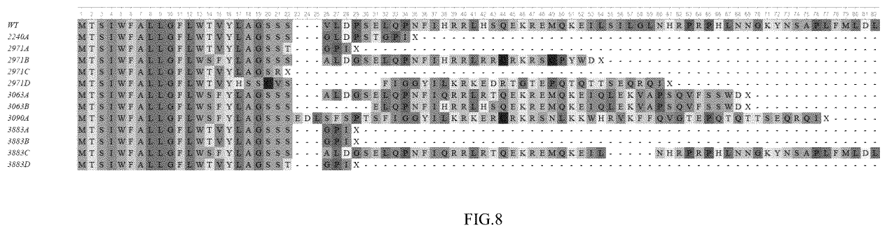

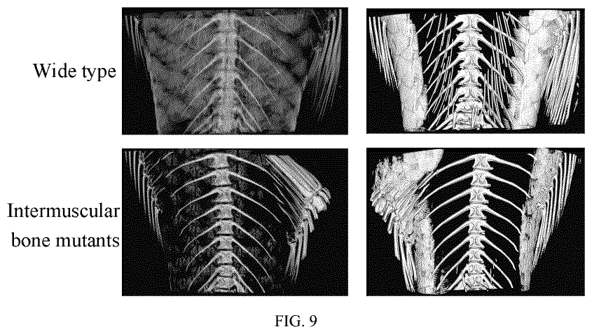

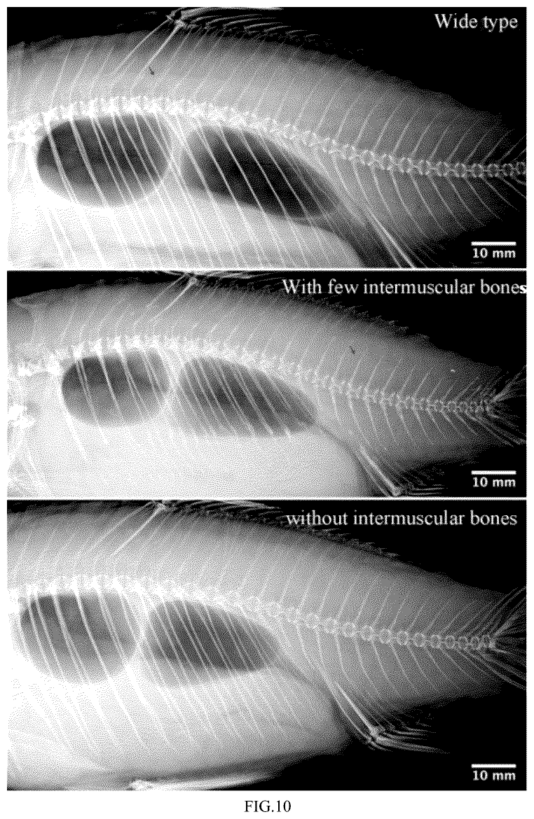

The bones of the new Carassius auratus strain with intermuscular bone-deficient in this example were observed by bone-staining, and it was found that the intermuscular bones were completely deficient in 5 samples, and the number of intermuscular bones in 4 samples was only 4 to 20 (as shown in FIG. 1 to 4, 9 and 10). The mutant alleles of above samples in exon 1 of bmp6a and exon 1 of bmp6b were detected, and it was found that mutations were observed in all three target sites of exon 1 (as shown in FIG. 5A to C and FIG. 6A to C). Pre-mature of stop codons in proteins was caused by gene knockout (as shown in FIGS. 7 and 8).

Although the present disclosure has been described in detail through the above example, it is only a part of embodiments of the present disclosure, rather than all embodiments. People can also obtain other embodiments according to the present embodiment without creativity. These embodiments all fall within the claimed scope of the present disclosure.

1. A method for breeding Carassius auratus strains without intermuscular bones, wherein the breeding of the Carassius auratus strains with intermuscular bone-deficient according to following steps:
designing knockout target sites for two copies of bmp6 gene in the Carassius auratusn genome, bmp6a and bmp6b, respectively, and then obtaining F2 generation individuals from a homozygous line with bmp6a and bmp6b double gene mutation through two rounds of gene knockout and screening, and then using the F2 generation individuals from the homozygous line with bmp6a and bmp6b double gene mutation to propagate to form a new Carassius auratus strain without intermuscular bones;
wherein, mixing a sgRNA corresponding to (bmp6a), (bmp6b) or (bmp6a and bmp6b) with Cas9 protein and microinjecting a mixture obtained into Carassius auratus embryos in the single cell stage, to perform a first round of gene knockout to construct a F0 generation population, culturing the F0 generation population for 3 to 5 months followed by PIT labeling and DNA extraction, then sequencing to determine alleles and mutation rates of somatic mutations of Carassius auratus, selecting F0 generation individuals with a somatic mutation rate of more than 95% as parents to prepare 0-generation fertilized eggs ;
mixing the sgRNAs corresponding to (bmp6a), (bmp6b) or (bmp6a and bmp6b) with Cas9 protein and microinjecting a mixture obtained into the 0-generation fertilized eggs to perform a second round of gene knockout to construct a F1 generation population, culturing the F1 generation population for 3 to 5 months followed by PIT labeling and DNA extraction, then sequencing to determine alleles and mutation rates of somatic mutations of Carassius auratus, selecting F1 generation individuals from a somatic bmp6a and bmp6b double gene mutant line with a somatic mutation rate of more than 95% as parents for reproduction to construct F2 generation, then selecting F2 generation individuals from a homozygous line with bmp6a and bmp6b double gene mutation.

2. The method for breeding Carassius auratus strains without intermuscular bones according to claim 1, wherein the knockout target sites of bmp6 gene are shown in Table 1,
TABLE 1

| | sgRNAs | Genome | | |
|---|---|---|---|---|
| Gene | Number | coordinates | Exon | Target sequence |
| bmp6a | CAA1 | NC_039266.1: | Exon1 | GGTAGCAGTTCTG |
| | | 5784476 | | CACTAGACGG |
| | | | | (SEQ ID NO: 1) |
| | CAA2 | NC_039266.1: | Exon1 | CCAGCGGAGGTTG |
| | | 5784523 | | CGGACTCAGG |
| | | | | (SEQ ID NO: 2) |
| | CAA3 | NC_039266.1: | Exon1 | GAAAGAGATTCTG |
| | | 5784562 | | TCCATACTGG |
| | | | | (SEQ ID NO: 3) |
| | CAA4 | NC_039266.1: | Exon2 | TCTTTATGGTGTCT |
| | | 5815487 | | TCTCTGTGG |
| | | | | (SEQ ID NO: 4) |
| | CAA5 | NC_039266.1: | Exon2 | GGCCTCTCCCTCT |
| | | 5815527 | | GGAATCTGGG |
| | | | | (SEQ ID NO: 5) |
| | CAA6 | NC_039266.1: | Exon2 | TGCAGAATTCAGG |
| | | 5815558 | | ATCTACAAGG |
| | | | | (SEQ ID NO: 6) |
| bmp6b | CAA7 | NC_039291.1: | Exon1 | GGCAGGTAGTAGT |
| | | 8127537 | | TCTGTACTGG |
| | | | | (SEQ ID NO: 7) |
| | CAA8 | NC_039291.1: | Exon1 | AGCCCAACTTCAT |
| | | 8127477 | | TCATCGGAGG |
| | | | | (SEQ ID NO: 8) |
| | CAA9 | NC_03929.1: | Exon1 | GAAAGAGATCCTG |
| | | 8127425 | | TCCATACTGG |
| | | | | (SEQ ID NO: 9) |
| | CAA10 | NC_03929.1: | Exon2 | GGCCTCTCCCTCT |
| | | 8097264 | | GGAATCTGGG |
| | | | | (SEQ ID NO: 10) |
| | CAA11 | NC_039291.1: | Exon2 | AGCAGAATTCAGG |
| | | 8097233 | | ATCTACAAGG |
| | | | | (SEQ ID NO: 11) |

3. The method for breeding Carassius auratus strains without intermuscular bones according to claim 2, wherein sgRNA forward primers designed for the knockout target sites of the bmp6 gene are shown in the table below,

| | sgRNAs | |
|---|---|---|
| Gene | Number | sgRNA forward primer |
| bmp6a | CAA1 | TTCTAATACGACTCACTATAGGTAGCAGTT |
| | | CTGCACTAGAGTTTTAGAGCTAGA |
| | | (SEQ ID NO: 12) |
| | CAA2 | TTCTAATACGACTCACTATAGCCAGCGGAG |
| | | GTTGCGGACTCGTTTTAGAGCTAGA |
| | | (SEQ ID NO: 13) |
| | CAA3 | TTCTAATACGACTCACTATAGGAAAGAGAT |
| | | TCTGTCCATACGTTTTAGAGCTAGA |
| | | (SEQ ID NO: 14) |
| | CAA4 | TTCTAATACGACTCACTATAGTCTTTATGG |
| | | TGTCTTCTCTGGTTTTAGAGCTAGA |
| | | (SEQ ID NO: 15) |
| | CAA5 | TTCTAATACGACTCACTATAGGCCTCTCCC |
| | | TCTGGAATCTGTTTTAGAGCTAGA |
| | | (SEQ ID NO: 16) |
| | CAA6 | TTCTAATACGACTCACTATAGTGCAGAATT |
| | | CAGGATCTACAGTTTTAGAGCTAGA |
| | | (SEQ ID NO: 17) |
| bmp6b | CAA7 | TTCTAATACGACTCACTATAGGCAGGTAGT |
| | | AGTTCTGTACGTTTTAGAGCTAGA |
| | | (SEQ ID NO: 18) |
| | CAA8 | TTCTAATACGACTCACTATAGAGCCCAACT |
| | | TCATTCATCGGGTTTTAGAGCTAGA |
| | | (SEQ ID NO: 19) |
| | CAA9 | TTCTAATACGACTCACTATAGGAAAGAGAT |
| | | CCTGTCCATACGTTTTAGAGCTAGA |
| | | (SEQ ID NO: 20) |
| | CAA10 | TTCTAATACGACTCACTATAGGCCTCTCCC |
| | | TCTGGAATCTGTTTTAGAGCTAGA |
| | | (SEQ ID NO: 21) |
| | CAA11 | TTCTAATACGACTCACTATAGAGCAGAATT |
| | | CAGGATCTACAGTTTTAGAGCTAGA |
| | | (SEQ ID NO: 22) |

4. The method for breeding Carassius auratus strains without intermuscular bones according to claim 3, wherein using a sgRNA forward primer and a sgRNA reverse primer with the sequence of 5′-GATCCGCACCGACTCGGTGCCACTTTTTCAAGTTGATAACGGACTAGCCTTATTTTA ACTTGCTATTTCTAGCTCTAAAAC-3′ (SEQ ID NO:31) to synthesis sgRNA in vitro; a sgRNA synthesis and amplification volume is: 2.5 μL of 10 μM sgRNA forward and reverse primers, 25 μL of 2×Dream Taq Master mix, and supplementing the sgRNA synthesis and amplification volume to 50 μL with enzyme-free water; sgRNA synthesis procedure in vitro is: denaturation at 95° C. for 3 min; then 30 cycles of 95° C. for 30 s, 58° C. for 30 s and 72° C. for 30 s, extension at 72° C. for 5 min;
after synthesizing the sgRNA in vitro, purifying and recovering an obtained sgRNA PCR product, then using an RNA in vitro transcription kit to transcribe the sgRNA in vitro; establishing a 30 μL reaction volume for sgRNA in vitro transcription for each target site: 1 μg of sgRNA PCR recovered product, 10 μL of NTP Buffer Mix, and 2 μL of T7 RNA Polymerase Mix, supplementing the sgRNA in vitro transcription reaction volume to 30 μL with enzyme-free water; transcribing at 37° C. for 4 h, adding 20 μL of enzyme-free water after the reaction, mixing well, adding 2 μL DNase I, digesting at 37° C. for 15 min to remove DNA.

5. The method for breeding Carassius auratus strains without intermuscular bones according to claim 4, wherein a method for the microinjection is: mixing the sgRNA synthesized in vitro and Cas9 protein in a molar concentration ratio of 3:1, then incubating at room temperature for 10 minutes, adding 25% phenol red and injecting the obtained product into Carassius auratus embryos in a single cell stage; wherein, mixing target site sgRNAs on each exon in equal amounts before injection, and a final concentration of each sgRNA is no less than 50 ng/μL, an injection volume of each fertilized egg is 1 nL±0.02 nL.

6. The method for breeding Carassius auratus strains without intermuscular bones according to claim 4, wherein a recovery concentration of the sgRNA PCR product is in a range of 100 to 160 ng/μL.

7. The method for breeding Carassius auratus strains without intermuscular bones according to claim 4, wherein a recovery concentration of the transcribed sgRNA in vitro is a range of 800 to 3000 ng/μL.

8. A Carassius auratus strain without intermuscular bones obtained by using the method for breeding Carassius auratus strains without intermuscular bones in any one of claims 1.

9. A reagent for knocking out the bmp6 gene in Carassius auratus genome, the reagent comprising sgRNA forward primers and sgRNA reverse primers designed for knockout target sites of bmp6 gene, the sgRNA forward primers and sgRNA reverse primers are shown in Table 1.

10. The reagent according to claim 9, wherein the sgRNA forward primers are shown in following table,

| | sgRNAs | |
|---|---|---|
| Gene | Number | sgRNA forward primer |
| bmp6a | CAA1 | TTCTAATACGACTCACTATAGGTAGCAGTTCT |
| | | GCACTAGAGTTTTAGAGCTAGA |
| | | (SEQ ID NO: 12) |
| | CAA2 | TTCTAATACGACTCACTATAGCCAGCGGAGGT |
| | | TGCGGACTCGTTTTAGAGCTAGA |
| | | (SEQ ID NO: 13) |
| | CAA3 | TTCTAATACGACTCACTATAGGAAAGAGATTC |
| | | TGTCCATACGTTTTAGAGCTAGA |
| | | (SEQ ID NO: 14) |
| | CAA4 | TTCTAATACGACTCACTATAGTCTTTATGGTG |
| | | TCTTCTCTGGTTTTAGAGCTAGA |
| | | (SEQ ID NO: 15) |
| | CAA5 | TTCTAATACGACTCACTATAGGCCTCTCCCTC |
| | | TGGAATCTGTTTTAGAGCTAGA |
| | | (SEQ ID NO: 16) |
| | CAA6 | TTCTAATACGACTCACTATAGTGCAGAATTCA |
| | | GGATCTACAGTTTTAGAGCTAGA |
| | | (SEQ ID NO: 17) |
| bmp6b | CAA7 | TTCTAATACGACTCACTATAGGCAGGTAGTAG |
| | | TTCTGTACGTTTTAGAGCTAGA |
| | | (SEQ ID NO: 18) |
| | CAA8 | TTCTAATACGACTCACTATAGAGCCCAACTTC |
| | | ATTCATCGGGTTTTAGAGCTAGA |
| | | (SEQ ID NO: 19) |
| | CAA9 | TTCTAATACGACTCACTATAGGAAAGAGATCC |
| | | TGTCCATACGTTTTAGAGCTAGA |
| | | (SEQ ID NO: 20) |
| | CAA10 | TTCTAATACGACTCACTATAGGCCTCTCCCTC |
| | | TGGAATCTGTTTTAGAGCTAGA |
| | | (SEQ ID NO: 21) |
| | CAA11 | TTCTAATACGACTCACTATAGAGCAGAATTCA |
| | | GGATCTACAGTTTTAGAGCTAGA |
| | | (SEQ ID NO: 22) |

11. The reagent according to claim 9, wherein the nucleotide sequence of the sgRNA reverse primer is: 5′-GATCCGCACCGACTCGGTGCCACTTTTTCAAGTTGATAACGGACTAGCCTTATTTTA ACTTGCTATTTCTAGCTCTAAAAC-3′ (SEQ ID NO:31).


SEQUENCE LISTING (31 sequences)

SEQ ID NO: 1 - DNA, 23 bp, Artificial Sequence
  Target sites sequence of sgRNA CAA1
ggtagcagttctgcactagacgg

SEQ ID NO: 2 - DNA, 23 bp, Artificial Sequence
  Target sites sequence of sgRNA CAA2
ccagcggaggttgcggactcagg

SEQ ID NO: 3 - DNA, 23 bp, Artificial Sequence
  Target sites sequence of sgRNA CAA3
gaaagagattctgtccatactgg

SEQ ID NO: 4 - DNA, 23 bp, Artificial Sequence
  Target sites sequence of sgRNA CAA4
tctttatggtgtcttctctgtgg

SEQ ID NO: 5 - DNA, 23 bp, Artificial Sequence
  Target sites sequence of sgRNA CAA5
ggcctctccctctggaatctggg

SEQ ID NO: 6 - DNA, 23 bp, Artificial Sequence
  Target sites sequence of sgRNA CAA6
tgcagaattcaggatctacaagg

SEQ ID NO: 7 - DNA, 23 bp, Artificial Sequence
  Target sites sequence of sgRNA CAA7
ggcaggtagtagttctgtactgg

SEQ ID NO: 8 - DNA, 23 bp, Artificial Sequence
  Target sites sequence of sgRNA CAA8
agcccaacttcattcatcggagg

SEQ ID NO: 9 - DNA, 23 bp, Artificial Sequence
  Target sites sequence of sgRNA CAA9
gaaagagatcctgtccatactgg

SEQ ID NO: 10 - DNA, 23 bp, Artificial Sequence
  Target sites sequence of sgRNA CAA10
ggcctctccctctggaatctggg

SEQ ID NO: 11 - DNA, 23 bp, Artificial Sequence
  Target sites sequence of sgRNA CAA11
agcagaattcaggatctacaagg

SEQ ID NO: 12 - DNA, 54 bp, Artificial Sequence
  Forward primer of sgRNA CAA1
ttctaatacgactcactataggtagcagttctgcactagagttttagagctaga

SEQ ID NO: 13 - DNA, 55 bp, Artificial Sequence
  Forward primer of sgRNA CAA2
ttctaatacgactcactatagccagcggaggttgcggactcgttttagagctaga

SEQ ID NO: 14 - DNA, 55 bp, Artificial Sequence
  Forward primer of sgRNA CAA3
ttctaatacgactcactataggaaagagattctgtccatacgttttagagctaga

SEQ ID NO: 15 - DNA, 55 bp, Artificial Sequence
  Forward primer of sgRNA CAA4
ttctaatacgactcactatagtctttatggtgtcttctctggttttagagctaga

SEQ ID NO: 16 - DNA, 54 bp, Artificial Sequence
  Forward primer of sgRNA CAA5
ttctaatacgactcactataggcctctccctctggaatctgttttagagctaga

SEQ ID NO: 17 - DNA, 55 bp, Artificial Sequence
  Forward primer of sgRNA CAA6
ttctaatacgactcactatagtgcagaattcaggatctacagttttagagctaga

SEQ ID NO: 18 - DNA, 54 bp, Artificial Sequence
  Forward primer of sgRNA CAA7
ttctaatacgactcactataggcaggtagtagttctgtacgttttagagctaga

SEQ ID NO: 19 - DNA, 55 bp, Artificial Sequence
  Forward primer of sgRNA CAA8
ttctaatacgactcactatagagcccaacttcattcatcgggttttagagctaga

SEQ ID NO: 20 - DNA, 55 bp, Artificial Sequence
  Forward primer of sgRNA CAA9
ttctaatacgactcactataggaaagagatcctgtccatacgttttagagctaga

SEQ ID NO: 21 - DNA, 54 bp, Artificial Sequence; identical to SEQ ID NO: 16
  Forward primer of sgRNA CAA10

SEQ ID NO: 22 - DNA, 55 bp, Artificial Sequence
  Forward primer of sgRNA CAA11
ttctaatacgactcactatagagcagaattcaggatctacagttttagagctaga

SEQ ID NO: 23 - DNA, 24 bp, Artificial Sequence
  Forward primer CAA-imb1a-E1
atcacagactggatgtaaagatga

SEQ ID NO: 24 - DNA, 19 bp, Artificial Sequence
  Reverse primer CAA-imb1a-E1
catgaagaggggtgctgaa

SEQ ID NO: 25 - DNA, 22 bp, Artificial Sequence
  Forward primer CAA-imb1a-E2
gttcgtcaagcacactaaaact

SEQ ID NO: 26 - DNA, 19 bp, Artificial Sequence
  Reverse primer CAA-imb1a-E2
ctttctggatgttccccaa

SEQ ID NO: 27 - DNA, 19 bp, Artificial Sequence
  Forward primer CAA-imb1b-E1
tccagcatgaagaggggtg

SEQ ID NO: 28 - DNA, 22 bp, Artificial Sequence
  Reverse primer CAA-imb1b-E1
ctggatgtaaagatgacaagca

SEQ ID NO: 29 - DNA, 19 bp, Artificial Sequence
  Forward primer CAA-imb1b-E2
acaggcagcagttgacttg

SEQ ID NO: 30 - DNA, 21 bp, Artificial Sequence
  Reverse primer CAA-imb1b-E2
gctttcaccacagagaagaca

SEQ ID NO: 31 - DNA, 81 bp, Artificial Sequence
  sgRNA reverse primer
gatccgcaccgactcggtgccactttttcaagttgataacggactagccttattttaacttgctatttctagctctaaaac